(19) United States
(12) Patent Application Publication
(10) Pub. No.: US 2009/0054658 A1
(43) Pub. Date: Feb. 26, 2009

(54) Liquid Crystal Compound, Liquid Crystal Composition, Thin Film and Liquid Crystal Display

(75) Inventor: Shigeki Uehira, Kanagawa (JP)

Correspondence Address: BUCHANAN, INGERSOLL & ROONEY PC, POST OFFICE BOX 1404, ALEXANDRIA, VA, 22313-1404, US

(73) Assignee: Fujifilm Corporation, Minato-ku (JP)

(21) Appl. No.: 11/988,361

(22) PCT Filed: Jul. 14, 2006
(86) PCT No.: PCT/JP2006/314436

§ 371 Date: Jan. 7, 2008

(30) Foreign Application Priority Data
Jul. 15, 2005 (JP) 2005-207143
Nov. 11, 2005 (JP) 2005-327001

(51) Int. Cl. C07D 271/06

(52) U.S. Cl. 548/131

(57) ABSTRACT

A liquid crystal compound is provided and includes a region in which an aromatic heterocyclic ring and an aromatic hydrocarbon ring linked together through a single bond, the liquid crystal compound expressing a nematic liquid crystal phase satisfying relational expression (1):

1.1≦(nx−nz)/(nx−ny)≦20

nx, ny and nz represent refractive indexes in three intersecting directions of the nematic liquid crystal phase, nx is the largest refractive index, and nz is the smallest refractive index.

### TECHNICAL FIELD

The invention relates to a liquid crystal compound which expresses a biaxial liquid crystal phase, a thin film which uses the liquid crystal compound and a liquid crystal display which uses the liquid crystal compound. In addition, the invention also relates to a liquid crystal compound having a specified structure useful in a retardation plate and the like, a thin film which uses the liquid crystal compound and a liquid crystal display which uses the liquid crystal compound.

### BACKGROUND ART

A biaxial film which controls a refractive index for each of triaxial directions is useful in the field of optical science which uses polarization. This is particularly useful in the field of liquid crystal display because of the aptness to control polarization.

A biaxial film prepared by making use of a biaxial liquid crystal compound has been reported (e.g., see JP-A-2002-6138). Also, an optical film prepared using a polymerizable group-introduced biaxial liquid crystal compound has been reported (e.g., see JP-A-2002-174730). However, once a polymerizable functional group is introduced into a biaxial liquid crystal compound, it is apt to cause a hybrid orientation (not biaxial). In addition, it is considered that orientation turbulence is also apt to occur due to increased pre-tilt angle of the liquid crystal compound molecule at the air interface. Accordingly, a liquid crystal compound having a biaxial liquid crystal phase suitable for biaxial films has been in demand.

It is known that refractive index anisotropy (Δn) generally becomes large as the temperature becomes low. An optical compensation film having further large refractive index anisotropy can be obtained by making use of a liquid crystal compound which can be polymerized at a low temperature. In addition, when polymerization can be carried out at a low temperature, it results in advantages such as the ability to simplify technical specifications of production facilities and/or to cut the production energy. Accordingly, a concern has been directed toward the development of a compound having a low transition temperature to the low temperature side phase.

### DISCLOSURE OF THE INVENTION

An object of an illustrative, non-limiting embodiment of the invention is to provide a liquid crystal compound having a biaxial liquid crystal phase, particularly capable of expressing a biaxial nematic liquid crystal phase, and a liquid crystal composition comprising the compound.

Another object of an illustrative, non-limiting embodiment of the invention is to provide a liquid crystal compound having a low transition temperature to the low temperature side phase, and a liquid crystal composition comprising the compound.

In addition, it is also an object to provide a retardation plate as a part of the use of these liquid crystal compounds and compositions.

The aforementioned problems can be solved by the following means.

(1) A liquid crystal compound comprising a region in which an aromatic heterocyclic ring and an aromatic hydrocarbon ring linked together through a single bond, the liquid crystal compound expressing a nematic liquid crystal phase satisfying relational expression (1):

1.1≦(nx−nz)/(nx−ny)≦20

wherein nx, ny and nz represent refractive indexes in three intersecting directions of the nematic liquid crystal phase, nx is the largest refractive index, and nz is the smallest refractive index.
(2) The liquid crystal compound described in the aforementioned (1), wherein the aromatic heterocyclic ring comprises at least one selected from the group consisting of 1,2,4-oxadiazole ring, 1,3,4-oxadiazole ring, 1,2,4-thiodiazole ring and 1,3,4-thiodiazole ring.
(3) The liquid crystal compound described in the aforementioned (1), wherein the aromatic heterocyclic ring is 1,2,4-oxadiazole ring.
(4) The liquid crystal compound described in the aforementioned (1), wherein the liquid crystal compound is a compound represented by one of formulae (1) and (2):

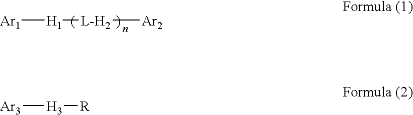

in the formula (1), Ar₁ and Ar₂ each independently represent a substituted or unsubstituted phenyl group, biphenyl group or naphthyl group; H₁ and H₂ each represent 1,2,4-oxadiazole ring; L represents a divalent connecting group, and n is an integer of 0 or 1, and

in the formula (2), Ar₃ represents a substituted or unsubstituted phenyl group, biphenyl group or naphthyl group; H₃ represents 1,2,4-oxadiazole ring; and R represents a substituted or unsubstituted alkyl group having from 1 to 20 carbon atoms.

(5) A liquid crystal compound expressing a nematic liquid crystal phase, which is represented by formula (3):

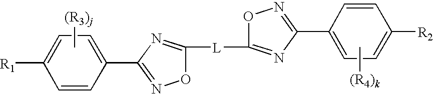

in the formula (3), L represents cyclohexyl or methaphenilene; R₁ to R₄ each independently represent a substituent group; and j and k each independently represent an integer of 0 to 4.
(6) A liquid crystal composition comprising a liquid crystal compound described in any one of the aforementioned (1) to (5).
(7) A thin film formed from a liquid crystal composition described in the aforementioned (6).
(8) A liquid crystal display comprising a liquid crystal compound described in any one of the aforementioned according to (1) to (5).

### ADVANTAGE OF THE INVENTION

According to an aspect of the invention, a liquid crystal composition which has a biaxial liquid crystal phase, particularly expresses a biaxial nematic liquid crystal phase, can be provided. Also, a retardation plate which uses the liquid crystal composition and has an optical anisotropy wherein the refractive index is controlled at a desired value can be provided. In addition, an elliptic polarizing plate which can quite minutely control polarization and a liquid crystal display having broad angle of visibility can be provided by the retardation plate.

Also, when a temperature of a compound of the invention having a specified structure (e.g., a compound represented by the formula (3)) is lowered from the nematic phase state, its transition temperature to a low temperature side phase (e.g., crystal phase, smectic phase or the like) is low. Thus, when the compound has a polymerizable substituent group, it becomes possible to effect polymerization at a low temperature. On the other hand, since it is known that refractive index anisotropy (Δn) becomes large as the temperature becomes low (e.g., see Ekisho Binran (Liquid Crystal Handbook) published by Maruzen, p. 203), low polymerization temperature can be attained by the use of a compound of the invention having low transition temperature to the low temperature side phase, and a compensating film having large refractive index anisotropy can be obtained. In addition, surface irregularity and the like occur sometimes in preparing compensating films, due to disturbance of orientation caused by the formation of fine crystals in the liquid crystal composition, but they can be solved by the use of the compound of the invention having low transition temperature to the low temperature side phase, so that it becomes possible to prepare a uniformly oriented compensating film.

### DETAILED DESCRIPTION OF THE INVENTION

Exemplary embodiments of the invention are described in detail. Explanations on the composing elements described in the following are made based on exemplary embodiments of the invention in many cases, but the invention is not limited to such embodiments. In this connection, the numerical range expressed using “-” means a range which includes numerical values before and after the “-” as the lower limit value and upper limit value.

### (Biaxial Liquid Crystal Compound)

A liquid crystal compound of the invention shows optically biaxial property. In other words, it is a liquid crystal compound in which the refractive indexes in three axial directions nx, ny and nz are different from one another and satisfy, for example, a relationship of nx>ny>nz.

It is desirable that the liquid crystal compound to be used in the invention has the aforementioned properties and, at the same time, shows good mono-domain property for uniform and perfect orientation. When the mono-domain property is poor, the obtained structure becomes poly-domain, and an orientation defect is formed at the border between domains, so that light scatters. This is not desirable because it also results in the reduction of transmittance of a retardation plate.

As the biaxial liquid crystal phase shown by the liquid crystal compound to be used in the invention, a biaxial nematic phase, a biaxial smectic phase A and a biaxial smectic phase C can be exemplified. Among these liquid crystal phases, a biaxial nematic phase (Nb phase) which shows good mono-domain property is desirable. The biaxial nematic phase is one of liquid crystal phases which cam be formed by a nematic liquid crystal compound, and is a state in which, when the space of the liquid crystal phase is defined by x axis, y axis and z axis, the liquid crystal compound is prohibited to take a free rotation of the xz plane with the y axis in the center and a free rotation of the xy plane with the z axis in the center.

When a liquid crystal composition of the invention expresses a biaxial liquid crystal phase, and when the refractive indexes of three directions of the biaxial liquid crystal phase are defined as nx, ny and nz (nx>ny>nz), it is desirable that respective values satisfy the following relational expression (1), and it is more desirable to satisfy the following relational expression (2). By satisfying the values of this range, angle-dependency of retardation can be controlled in response to the liquid crystal display.

1.1≦(nx−nz)/(nx−ny)≦20 Relational expression (1)

1.2≦(nx−nz)/(nx−ny)≦10 Relational expression (2)

It is desirable that a liquid crystal composition of the invention expresses the liquid crystal phase within a range of from −100° C. to 300° C., more preferably from −50° C. to 280° C., most preferably from −40° C. to 250° C. The term “expresses the liquid crystal phase at from −100° C. to 300° C.” as used herein also includes a case in which the liquid crystal temperature range stands astride −100° C. (e.g., illustratively from −120° C. to −90° C.) and a case in which it stands astride 300° C. (e.g., illustratively from 298° C. to 310° C.). This is the same regarding from −50° C. to 280° C. and from −40° C. to 250° C., too.

In the case of the preparation of thin films, it is desirable that the aforementioned liquid crystal compound to be used in the invention is a polymerizable compound and/or a high molecular compound. The polymerizable compound may be a low molecular compound or a high molecular compound. In the case of a high molecular compound, this is desirably a polymerizable compound for carrying out fixing of orientation, but is not always necessarily polymerizable when the glass transition point is 30° C. or more.

As the low molecular liquid crystal compound having a polymerizable group, the compounds described in JP-A-2002-174730 can for example be used. However, when a compound which is apt to cause hybrid orientation or a compound which is apt to cause orientation turbulence due to increased pre-tilt angle of the molecule at the air interface, like the compounds described in this document, is used as the low molecular compound, it is desirable to add an air interface orientation controlling agent which is described later.

Regarding the liquid crystal compound which satisfies the aforementioned relational expression (1) and expresses a nematic phase comprising a region wherein an aromatic heterocyclic ring and an aromatic hydrocarbon ring are linked together through a single bond, it is desirable that the aromatic heterocyclic ring comprises at least one species selected from the group consisting of 1,2,4-oxadiazole ring, 1,3,4-oxadiazole ring, 1,2,4-thiodiazole ring and 1,3,4-thiodiazole ring, and it is more desirable that the aromatic heterocyclic ring is 1,2,4-oxadiazole ring. Illustratively, it is desirable that this compound is a compound represented by the following formula (1) or the following formula (2).

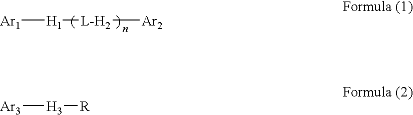

In the formula (1), Ar₁ and Ar₂ each independently represent a substituted or unsubstituted phenyl group, biphenyl group or naphthyl group, which may be the same or different from each other, H₁ and H₂ each represent 1,2,4-oxadiazole ring, L represents a divalent connecting group, and n is an integer of 0 or 1.

In the formula (2), Ar₃ represents a substituted or unsubstituted phenyl group, biphenyl group or naphthyl group, H₃ represents 1,2,4-oxadiazole ring, and R represents a substituted or unsubstituted alkyl group having from 1 to 20 carbon atoms.

The following can be cited as preferred examples of the substituent groups which may be possessed by Ar₁ and Ar₂.

A halogen atom (e.g., fluorine atom, chlorine atom, bromine atom or iodine atom), an alkyl group (preferably an alkyl group having from 1 to 30 carbon atoms, such as methyl group, ethyl group, n-propyl group, isopropyl group, t-butyl group, n-octyl group or 2-ethylhexyl group), a cycloalkyl group (preferably a substituted or unsubstituted cycloalkyl group having from 3 to 30 carbon atoms, such as cyclohexyl group, cyclopentyl group or 4-n-dodecylcyclohexyl group), a bicycloalkyl group (preferably a substituted or unsubstituted bicycloalkyl group having from 5 to 30 carbon atoms, namely a monovalent group in which one hydrogen atom is removed from a bicycloalkane having from 5 to 30 carbon atoms, such as bicyclo(1,2,2)heptan-2-yl or bicyclo(2,2,2)octan-2-yl), an alkenyl group (preferably a substituted or unsubstituted alkenyl group having from 2 to 30 carbon atoms, such as vinyl group or allyl group), a cycloalkenyl group (preferably a substituted or unsubstituted cycloalkenyl group having from 3 to 30 carbon atoms, namely a monovalent group in which one hydrogen atom is removed from a cycloalkane having from 3 to 30 carbon atoms, such as 2-cyclopenten-1-yl or 2-cyclohexen-1-yl), a bicycloalkenyl group (a substituted or unsubstituted bicycloalkenyl group, preferably a substituted or unsubstituted bicycloalkenyl group having from 5 to 30 carbon atoms, namely a monovalent group in which one hydrogen atom is removed from a bicycloalkane having one double bond, such as bicyclo(2,2,1)hept-2-en-1-yl or bicyclo(2,2,2)oct-2-en-4-yl), an alkynyl group (preferably a substituted or unsubstituted alkynyl group having from 2 to 30 carbon atoms, such as ethynyl group or propargyl group), an aryl group (preferably a substituted or unsubstituted aryl group having from 6 to 30 carbon atoms, such as phenyl group, p-tolyl group or naphthyl group), a heterocyclic group (preferably a monovalent group in which one hydrogen atom is removed from a 5- or 6-membered substituted or unsubstituted aromatic or non-aromatic heterocyclic compound, more preferably a 5- or 6-membered aromatic heterocyclic group having from 3 to 30 carbon atoms, such as 2-furyl group, 2-thienyl group, 2-pyrimidinyl group or 2-benzothiazolyl group), cyano group, hydroxyl group, nitro group, carboxyl group, an alkoxy group (preferably a substituted or unsubstituted alkoxy group having from 1 to 30 carbon atoms, such as methoxy group, ethoxy group, isopropoxy group, t-butoxy group, n-octyloxy group or 2-methoxyethoxy group), an aryloxy group (preferably a substituted or unsubstituted aryloxy group having from 6 to 30 carbon atoms, such as phenoxy group, 2-methylphenoxy group, 4-tert-butylphenoxy group, 3-nitrophenoxy group or 2-tetradecanoylaminophenoxy group), a silyloxy group (preferably a silyloxy group having from 3 to 20 carbon atoms, such as trimethylsilyloxy group or tert-butyldimethylsilyloxy group), a heterocyclic oxy group (preferably a substituted or unsubstituted heterocyclic oxy group having from 2 to 30 carbon atoms, such as 1-phenyltetrazole-5-oxy group or 2-tetrahydropyranyloxy group), an acyloxy group (preferably formyloxy group, a substituted or unsubstituted alkylcarbonyloxy group having from 6 to 30 carbon atoms, such as formyloxy group, acetyloxy group, pivaloyloxy group, stearoyloxy group, benzoyloxy group or p-methoxyphenylcarbonyloxy group), a carbamoyloxy group (preferably a substituted or unsubstituted carbamoyloxy group having from 1 to 30 carbon atoms, such as N,N-dimethylcarbamoyloxy group, N,N-diethylcarbamoyloxy group, morpholinocarbamoyloxy group, N,N-di-n-octylaminocarbamoyloxy group or N-n-octylcarbamoyloxy group), an alkoxycarbonyloxy group (preferably a substituted or unsubstituted alkoxycarbonyloxy group having from 2 to 30 carbon atoms, such as methoxycarbonyloxy group, ethoxycarbonyloxy group, tert-butoxycarbonyloxy group or n-octylcarbonyloxy group), an aryloxycarbonyloxy group (preferably a substituted or unsubstituted aryloxycarbonyloxy group having from 7 to 30 carbon atoms, such as phenoxycarbonyloxy group, p-methoxyphenoxycarbonyloxy group or p-n-hexadecyloxyphenoxycarbonyloxy group), an amino group (preferably amino group, a substituted or unsubstituted alkylamino group having from 1 to 30 carbon atoms or a substituted or unsubstituted anilino group having from 6 to 30 carbon atoms, such as amino group, methylamino group, dimethylamino group, anilino group, N-methyl-anilino group or diphenylamino group), an acylamino group (preferably formylamino group, a substituted or unsubstituted alkylcarbonylamino group having from 1 to 30 carbon atoms or a substituted or unsubstituted arylcarbonylamino group having from 6 to 30 carbon atoms, such as formylamino group, acetylamino group, pivaloylamino group, lauroylamino group or benzoylamino group), an aminocarbonylamino group (preferably a substituted or unsubstituted aminocarbonylamino group having from 1 to 30 carbon atoms, such as carbonylamino group, N,N-dimethylaminocarbonylamino group, N,N-diethylaminocarbonylamino group or morpholinocarbonylamino group), an alkoxycarbonylamino group (preferably a substituted or unsubstituted alkoxycarbonylamino group having from 2 to 30 carbon atoms, such as methoxycarbonylamino group, ethoxycarbonylamino group, tert-butoxycarbonylamino group, n-octadecyloxycarbonylamino group or N-methyl-methoxycarbonylamino group), an aryloxycarbonylamino group (preferably a substituted or unsubstituted aryloxycarbonylamino group having from 7 to 30 carbon atoms, such as phenoxycarbonylamino group, p-chlorophenoxycarbonylamino group or m-n-octyloxyphenoxycarbonylamino group), a sulfamoylamino group (preferably a substituted or unsubstituted sulfamoylamino group having from 0 to 30 carbon atoms, such as sulfamoylamino group, N,N-dimethylaminosulfonylamino group or N-n-octylaminosulfonylamino group), an alkyl or aryl sulfonylamino group (preferably a substituted or unsubstituted alkylsulfonylamino group having from 1 to 30 carbon atoms or a substituted or unsubstituted arylsulfonylamino group having from 6 to 30 carbon atoms, such as methylsulfonylamino group, butylsulfonylamino group, phenylsulfonylamino group, 2,3,5-trichlorophenylsulfonylamino group or p-methylphenylsulfonylamino group), mercapto group, an alkylthio group (preferably a substituted or unsubstituted alkylthio group having from 1 to 30 carbon atoms, such as methylthio group, ethylthio group or n-hexadecylthio group), an arylthio group (preferably a substituted or unsubstituted arylthio group having from 6 to 30 carbon atoms, such as phenylthio group, o-chloro phenylthio group or m-methoxy phenylthio group), a heterocyclic thio group (preferably a substituted or unsubstituted heterocyclic thio group having from 2 to 30 carbon atoms, such as 2-benzothiazolylthio group or 1-phenyl tetrazol-5-ylthio group), a sulfamoyl group (preferably a substituted or unsubstituted sulfamoyl group having from 0 to 30 carbon atoms, such as N-ethylsulfamoyl group, N-(3-dodecyloxypropyl)sulfamoyl group, N,N-dimethylsulfamoyl group, N-acetylsulfamoyl group, N-benzoylsulfamoyl group or N—(N′phenylcarbamoyl)sulfamoyl group), sulfo group, an alkyl or aryl sulfinyl group (preferably a substituted or unsubstituted alkyl sulfinyl group having from 1 to 30 carbon atoms or a substituted or unsubstituted aryl sulfinyl group having from 6 to 30 carbon atoms, such as methylsulfinyl group, ethylsulfinyl group, phenylsulfinyl group or p-methylphenylsulfinyl group), an alkyl or aryl sulfonyl group (preferably a substituted or unsubstituted alkyl sulfonyl group having from 1 to 30 carbon atoms or a substituted or unsubstituted aryl sulfonyl group having from 6 to 30 carbon atoms, such as methylsulfonyl group, ethylsulfonyl group phenylsulfonyl group or p-methylphenylsulfonyl group), an acyl group (preferably formyl group or a substituted or unsubstituted alkyl carbonyl group having from 2 to 30 carbon atoms or a substituted or unsubstituted aryl carbonyl group having from 7 to 30 carbon atoms, such as acetyl group or pivaloylbenzoyl group), an aryloxycarbonyl group (preferably a substituted or unsubstituted aryloxycarbonyl group having from 7 to 30 carbon atoms, such as phenoxycarbonyl group, o-chlorophenoxycarbonyl group, m-nitrophenoxycarbonyl group or p-tert-butylphenoxycarbonyl group), an alkoxycarbonyl group (preferably a substituted or unsubstituted alkoxycarbonyl group having from 2 to 30 carbon atoms, such as methoxy carbonyl group, ethoxy carbonyl group, tert-butoxy carbonyl group or n-octadecyloxy carbonyl group), a carbamoyl group (preferably a substituted or unsubstituted carbamoyl group having from 1 to 30 carbon atoms, such as carbamoyl group, N,N-di-n-octylcarbamoyl group or N-(methylsulfonyl)carbamoyl group), an aryl or heterocyclic azo group (preferably a substituted or unsubstituted arylazo group having from 6 to 30 carbon atoms or a substituted or unsubstituted heterocyclic azo group having from 3 to 30 carbon atoms, such as phenylazo group, p-chlorophenylazo group or 5-ethylthio-1,3,4-thiadiazol-2-ylazo group), an imido group (preferably N-succinimido group or N-phthalimido group), a phosphino group (preferably a substituted or unsubstituted phosphino group having from 2 to 30 carbon atoms, such as dimethylphosphino group, diphenylphosphino group or methylphenoxyphosphino group), a phosphinyl group (preferably a substituted or unsubstituted phosphinyl group having from 2 to 30 carbon atoms, such as phosphinyl group, dioctyloxyphosphinyl group or diethoxyphosphinyl group), a phosphinyloxy group (preferably a substituted or unsubstituted phosphinyloxy group having from 2 to 30 carbon atoms, such as diphenoxyphosphinyloxy group or dioctyloxyphosphinyloxy group), a phosphinylamino group (preferably a substituted or unsubstituted phosphinylamino group having from 2 to 30 carbon atoms, such as dimethoxyphosphinylamino group or dimethylaminophosphinylamino group) and a silyl group (preferably a substituted or unsubstituted silyl group having from 3 to 30 carbon atoms, such as trimethylsilyl group, tert-butyldimethylsilyl group or phenyldimethylsilyl group).

Among the aforementioned substituent groups, those which have a hydrogen atom may be freed from the hydrogen atom and further substituted with the aforementioned groups. Examples of such functional groups include an alkylcarbonylaminosulfonyl group, an arylcarbonylaminosulfonyl group, an alkylsulfonylaminocarbonyl group and an arylsulfonylaminocarbonyl group. Their examples include methylsulfonylaminocarbonyl group, p-methylphenylsulfonylaminocarbonyl group, acetylaminosulfonyl group and benzoylaminosulfonyl group.

More preferred are an alkyl group, an alkoxy group and an alkoxycarbonyl group, each having from 1 to 20 carbon atoms, and an alkoxycarbonyloxy group, a cyano group and a halogen atom.

H₁ and H₂ each represent 1,2,4-oxadiazole ring. The 3-position and the 5-position of the 1,2,4-oxadiazole ring are possible as the binding cite of H₁ and H₂ with Ar₁ and Ar₂, but they are not discriminated herein and may be the same or different.

L represents a divalent connecting group. L may have a substituent group. As examples of this substituent group, the aforementioned examples of substituent groups which may be possessed by Ar₁ and Ar₂ can be cited.

The following illustrative examples can be cited as preferred examples of L.

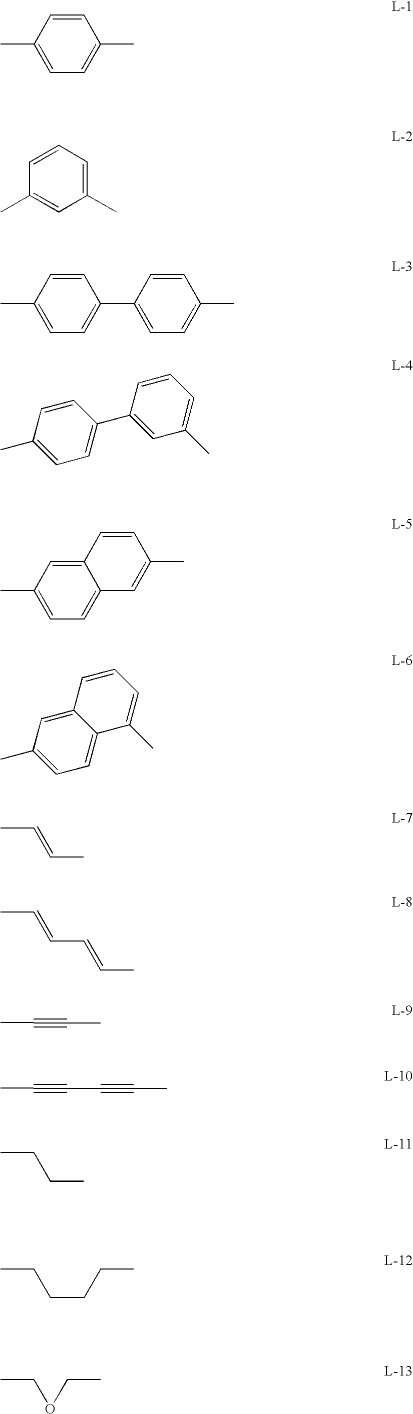

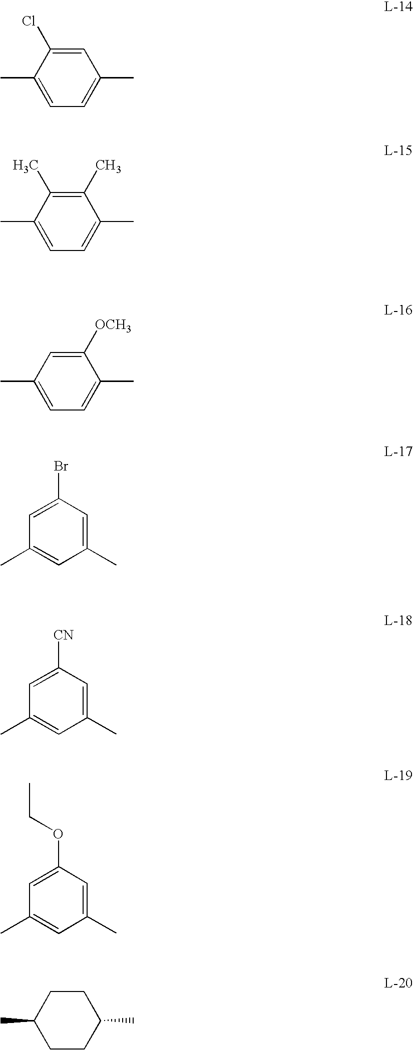

More preferred are phenylene (L-1 and L-2), biphenylene (L-3), naphthalene (L-5), cyclohexylene (L-20), ethylene (L-7) and acetylene (L-9).

The symbol n is an integer of 0 or 1.

Ar₃ represents a substituted or unsubstituted phenyl group, biphenyl group or naphthyl group. Its examples include those which were exemplified in the foregoing regarding Ar₁ and Ar₂.

H₃ represents 1,2,4-oxadiazole ring. The 3-position and the 5-position of the 1,2,4-oxadiazole ring are possible as the binding cite of H₃ with Ar₃, but they are not discriminated herein and may be the same or different.

R represents a substituted or unsubstituted alkyl group having from 1 to 20 carbon atoms. More preferred is a substituted or unsubstituted alkyl group having from 1 to 15 carbon atoms, and its examples include a butyl group, hexyl group, cyclohexyl group, 2-ethylhexyl group, octyl group and the like.

(Compound Represented by the Formula (3))

According to an aspect of the invention, a liquid crystal compound which expresses a nematic phase represented by the following formula (3) can be exemplified as the liquid crystal compound having low transition temperature to the low temperature side phase.

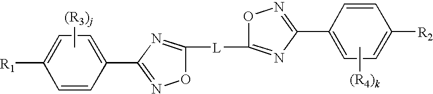

In the formula (3), L represents cyclohexyl or methaphenilene, R₁ to R₄ each independently represent a substituent groups, and j and k each independently represents an integer of from 0 to 4.

R₁ to R₄ each independently represents a substituent group, and the preferred examples of the aforementioned substituent group which may be possessed by Ar₁ and Ar₂ can be illustratively cited.

R₁ and R₂ each are preferably hydrogen atom, an alkyl group, an alkoxy group, a carbonyloxy group, an alkoxycarbonyl group or an alkoxycarbonyloxy group. R₁ and R₂ may be the same or different from each other. Preferred is R₁=R₂.

R₃ and R₄ are preferably an alkyl group, an alkoxy group, a carbonyloxy group, an alkoxycarbonyl group, an alkoxycarbonyloxy group or a halogen atom.

Illustrative examples of the compounds represented by the formula (1) to formula (3) are shown in the following, but the invention is not limited thereto. Regarding the following compounds, illustration compound (X) is shown by the number in the parentheses ( ) unless otherwise noted.

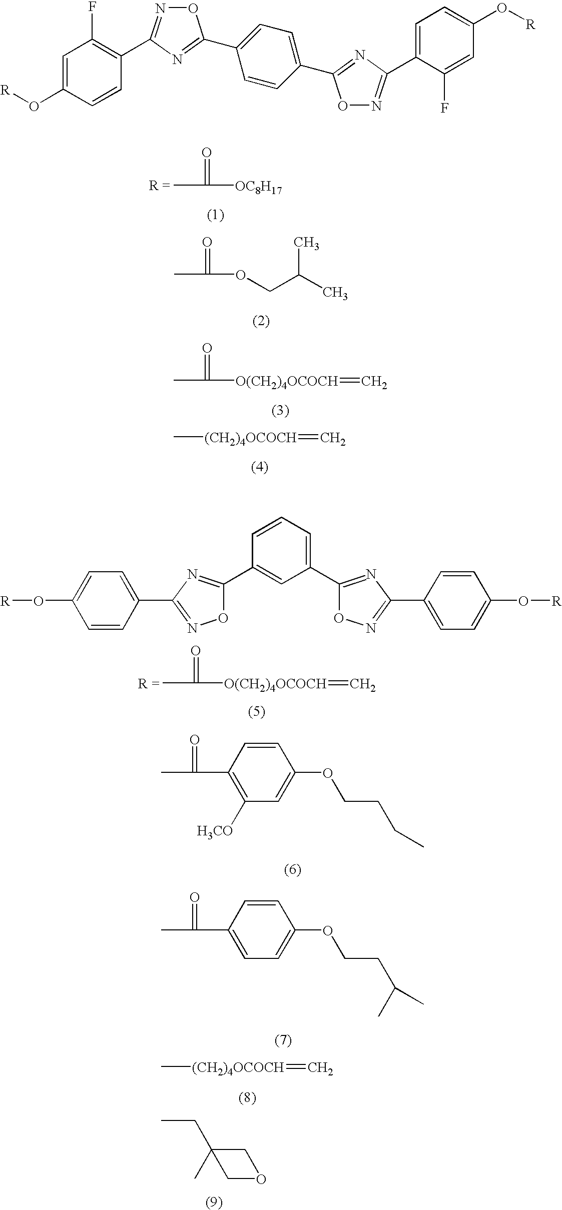

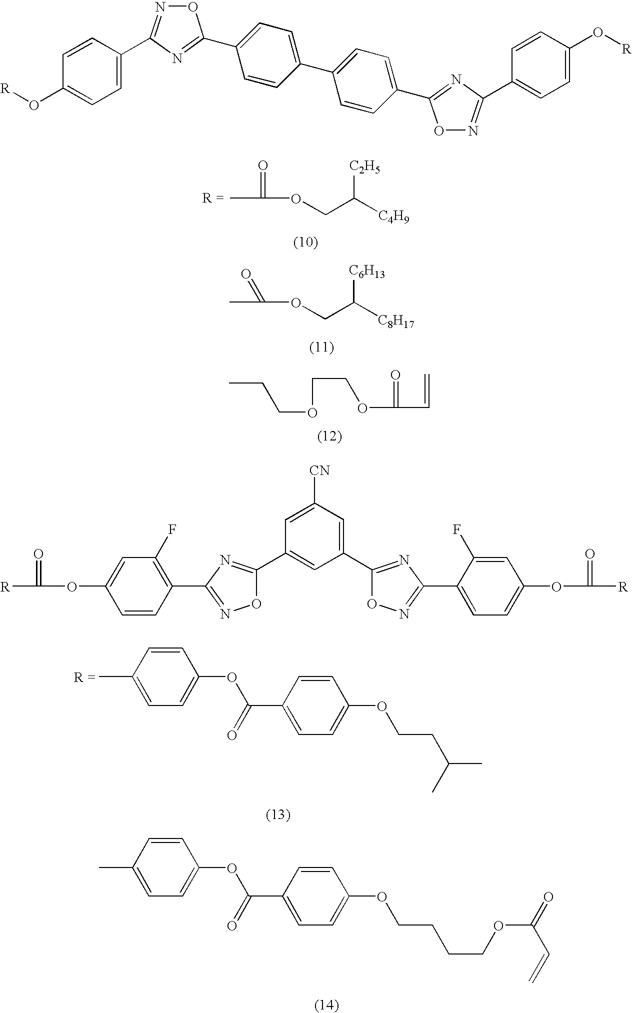

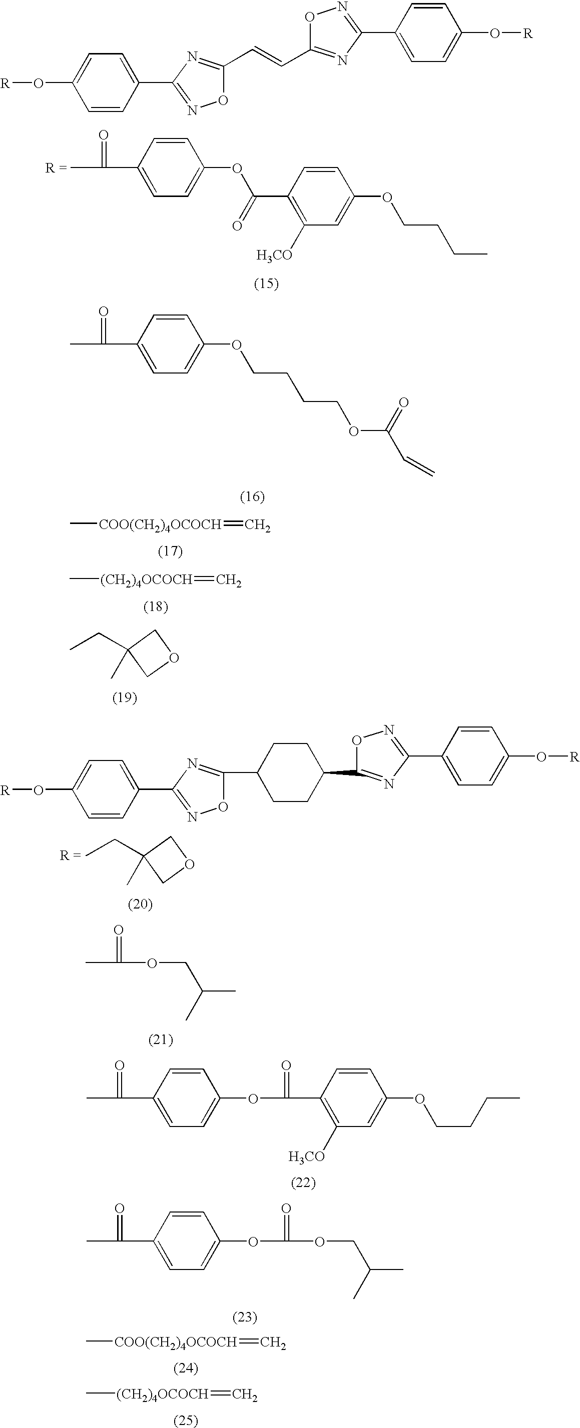

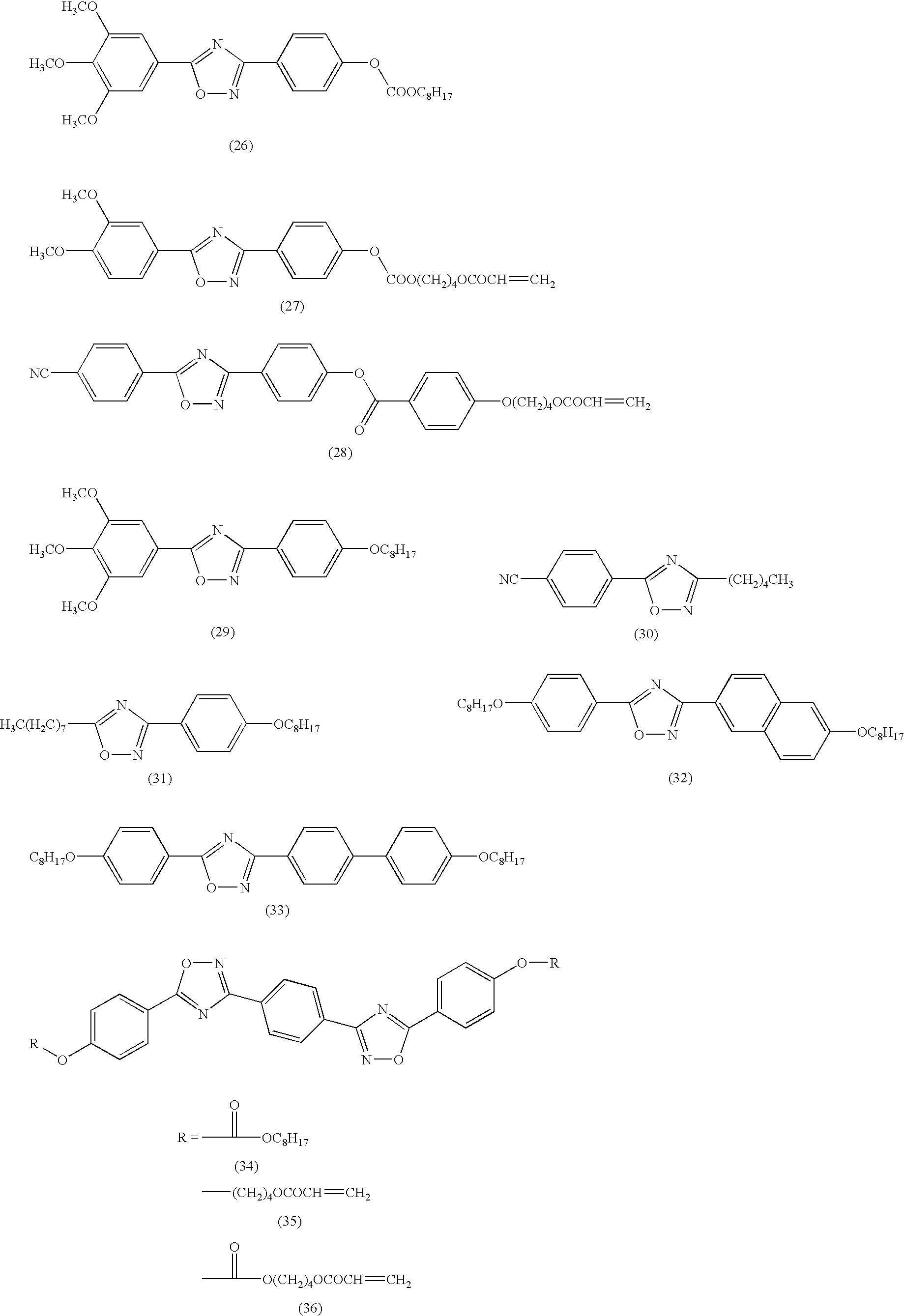

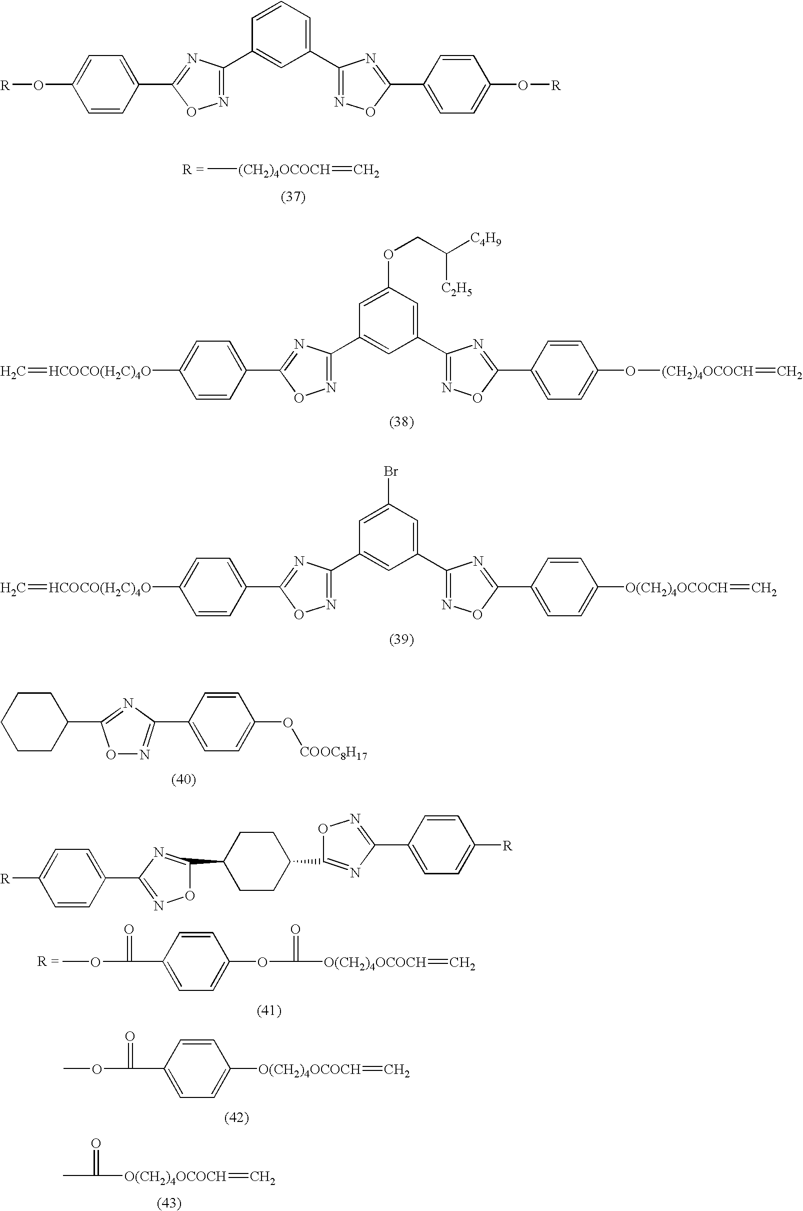

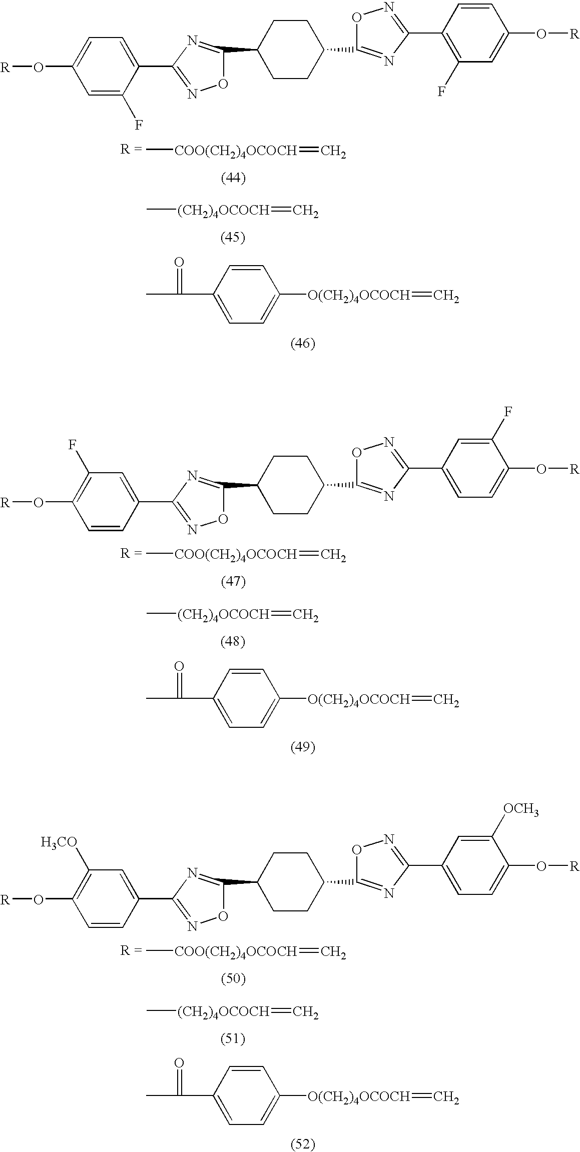

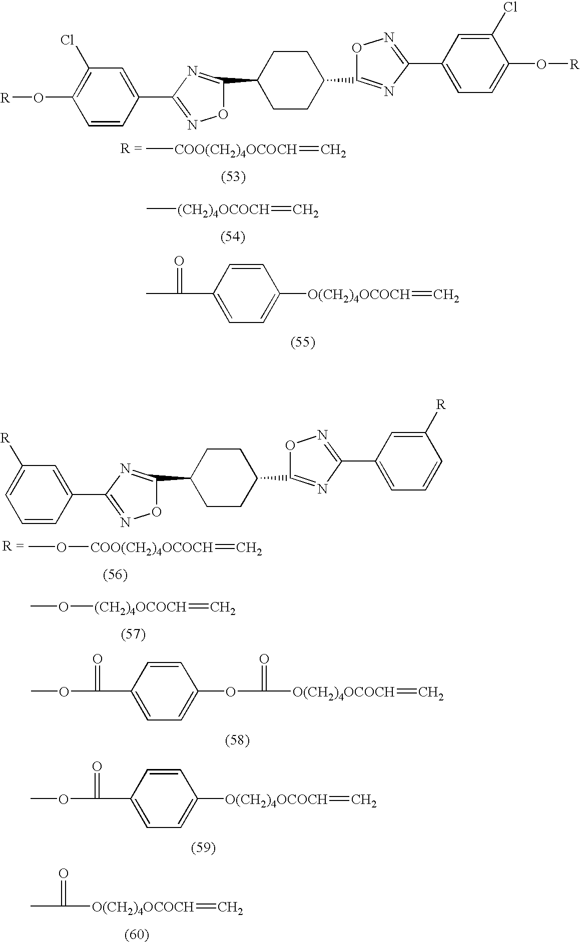

In order to realize the state in which a biaxial liquid crystal compound of the invention is uniformly oriented, it is desirable to prepare an orientation film. However, when the optical axis direction of a discotic liquid crystal coincides with the normal line direction of a thin film (homeotropic orientation), the orientation film is not always necessary.

The orientation film can be prepared by a certain means such as rubbing treatment of an organic compound (preferably a polymer), oblique deposition of an inorganic compound, formation of a layer having microgroove, or accumulation of an organic compound (e.g., ω-tricosanoic acid or methyl stearylate) by Langmuir-Blodgett technique (LB film). In addition, an orientation film in which an orientation function is generated by the application of electric field, application of magnetic field or light irradiation is also known.

The orientation film may be any layer, with the proviso that it can add a desired orientation to a liquid crystal composition of the invention, but an oriented film formed by a rubbing treatment or light irradiation is desirable in the case of the invention. An orientation film formed by a rubbing treatment of a polymer is particularly desirable. The rubbing treatment can be generally carried out by rubbing the surface of a polymer layer with paper or cloth several times toward a certain direction, but particularly in the invention, it is desirable to carry out it by the method described in Ekisho Binran (Liquid Crystal Handbook) published by Maruzen. It is desirable that thickness of the orientation film is from 0.01 to 10 μm, more desirably from 0.05 to 3 μm.

The state in which the orientation condition is fixed is the most typical and desired embodiment of the state in which the orientation is maintained, but not limited only to this, and it illustratively means a state in which the fixed liquid crystal composition does not have fluidity and can continuously and stably maintain the fixed orientation form without changing the orientation form by external field and external force, within the temperature range of generally from 0° C. to 50° C., or under a more severe condition of from −30° C. to 70° C. In this connection, once an optical anisotropy layer is formed through the final fixation of the orientation state, it is no longer necessary for the liquid crystal composition of the invention to show liquid crystal property. For example, since a compound having a polymerizable group is used as the liquid crystal compound, the liquid crystal property could be lost through the advance of polymerization or crosslinking reaction due to reactions caused by heat, light and the like as a result thereof and subsequent increase of molecular weight. As examples of the additive agent which can be added to the liquid crystal composition of the invention in forming the optical anisotropy layer, an air interface orientation controlling agent, a repelling inhibitor, a polymerization initiator, a polymerizable monomer and the like can be cited.

### (Air Interface Orientation Controlling Agent)

At the air interface, a liquid crystal composition is oriented at a tilt angle of the air interface. Since the degree of this tilt angle varies depending on the kind of liquid crystal compound, the kind of additive agent and the like contained in the liquid crystal composition, it is necessary to optionally control the tilt angle at the air interface in response to each purpose.

In order to control the aforementioned tilt angle, an electric field, magnetic field or the like external force or an additive agent can for example be used, but it is desirable to use an additive agent. As such an additive agent, a compound having one or more of a substituted or unsubstituted aliphatic group having from 6 to 40 carbon atoms, or a substituted or unsubstituted oligosiloxane group having from 6 to 40 carbon atoms, in the molecule is desirable, and a compound having two or more of the group in the molecule is more desirable. For example, as the air interface orientation controlling agent, the hydrophobic excluded volume effect compound described in JP-A-2002-20363 can be used.

Amount of the additive agent to be added for controlling the air interface side orientation is preferably from 0.001% by mass (or weight) to 20% by mass, more preferably from 0.01% by mass to 10% by mass, most preferably from 0.1% by mass to 5% by mass, based on the liquid crystal composition of the invention.

### (Repelling Inhibitor)

As a material for preventing a liquid crystal composition from being repelled at the time of applying the liquid crystal composition by adding to the composition, a high molecular compound can be suitably used in general.

The polymer to be used is not particularly limited, with the proviso that it does not considerably inhibit tilt angle change and orientation of the liquid crystal composition of the invention.

As examples of the polymer, they are described in JP-A-8-95030, and cellulose esters can be cited as illustrative examples of the particularly desirable polymer. As examples of the cellulose ester, cellulose acetate, cellulose acetate propionate, hydroxypropyl cellulose and cellulose acetate butyrate can be cited.

In order to prevent inhibition of orientation of a liquid crystal composition of the invention, amount of the polymer to be used for the purpose of inhibiting repelling is generally within the range of from 0.1 to 10% by mass, more preferably within the range of from 0.1 to 8% by mass, further preferably within the range of from 0.1 to 5% by mass, based on the liquid crystal composition of the invention.

### (Polymerization Initiator)

Regarding fixation of the oriented state according to the invention, a liquid crystal composition can be formed by once heating it to the liquid crystal phase forming temperature and then cooling it while keeping the oriented state, thereby effecting the fixation without spoiling the oriented form under the liquid crystal state. In addition, it can also be formed by heating a composition, prepared by adding an initiator to a liquid crystal composition of the invention, to the liquid crystal phase forming temperature and then polymerizing and cooling, thereby fixing the oriented form of the liquid crystal state. According to an aspect of the invention, it is desirable to carry out fixation of the oriented state by the latter polymerization reaction. A heat polymerization reaction which uses a heat polymerization initiator, a photopolymerization reaction which uses a photopolymerization initiator and a polymerization reaction by electron beam irradiation are included in the polymerization reaction, but the photopolymerization reaction and the polymerization reaction by electron beam irradiation are desirable for the purpose of preventing deformation and deterioration of the base material and the like by heat.

Examples of the photopolymerization initiator include a-carbonyl compounds (described in the respective specifications of U.S. Pat. No. 2,367,661 and U.S. Pat. No. 2,367,670), acyloin ethers (described in the specification of U.S. Pat. No. 2,448,828), α-hydrocarbon substituted aromatic acyloin compounds (described in the specification of U.S. Pat. No. 2,722,512), polynuclear quinone compounds (described in the specifications of U.S. Pat. No. 3,046,127 and U.S. Pat. No. 2,951,758), combination of triaryl imidazole dimer and p-aminophenyl ketone (described in the specification of U.S. Pat. No. 3,549,367), acridine and phenazine compounds (described in JP-A-60-105667 and in the specification of U.S. Pat. No. 4,239,850) and oxadiazole compounds (described in the specification of U.S. Pat. No. 4,212,970).

Amount of the photopolymerization initiator to be used is preferably from 0.01 to 20% by mass, more preferably from 0.5 to 5% by mass, based on the solid matter of application liquid of the optical anisotropy layer.

It is desirable to use ultraviolet rays in the light irradiation for polymerization. The irradiation energy is preferably from 10 mJ to 50 J/cm², more preferably from 50 mJ to 800 mJ/cm². In order to accelerate the photopolymerization reaction, the light irradiation may be carried out under a heating condition. In addition, since the concentration of oxygen in the atmosphere takes part in the polymerization degree, when desired polymerization degree cannot be obtained in the air, it is desirable to reduce the oxygen concentration by a nitrogen substitution or the like method. As the desirable oxygen concentration, 10% or less is preferable, 7% or less is more preferable, and 3% or less is most preferable.

### (Polymerizable Monomer)

A polymerizable monomer may be added to a liquid crystal composition of the invention. The polymerizable monomer which can be used in the invention is not particularly limited, with the proviso that it has compatibility with a compound of the invention and does not considerably cause orientation inhibition of the liquid crystal composition. Among such monomers, compounds having polymerization active ethylenic unsaturated groups, such as vinyl group, vinyloxy group, acryloyl group and methacryloyl group, are suitably used. Amount of the aforementioned polymerizable monomer to be added is generally within the range of from 0.5 to 50% by mass, preferably within the range of from 1 to 30% by mass. In addition, the use of a monomer in which the number of reactive functional groups is 2 or more is particularly desirable, because an effect to increase adhesion between the orientation film and the optical anisotropy layer can be expected.

### (Application Solvent)

An organic solvent is suitably used as the solvent to be used in the preparation of a liquid crystal composition of the invention. Examples of the organic solvent include an amide (e.g., N,N-dimethylformamide), a sulfoxide (e.g., dimethyl sulfoxide), a heterocyclic compound (e.g., pyridine), a hydrocarbon (e.g., toluene or hexane), an alkyl halide (e.g., chloroform or dichloromethane), an ester (e.g., methyl acetate or butyl acetate), a ketone (e.g., acetone, methyl ethyl ketone, methyl isobutyl ketone or cyclohexanone) and an ether (e.g., tetrahydrofuran or 1,2-dimethoxyethane). Two or more species of organic solvents may be used concomitantly.

### (Application Method)

A thin film of the invention is formed by preparing an application liquid of the liquid crystal composition of the invention using the aforementioned solvent, and applying it to the orientation film, thereby effecting orientation treatment of the liquid crystal composition of the invention. Application of the application liquid can be carried out by a conventionally known method (e.g., wire bar coating, extrusion coating, direct gravure coating, reverse gravure coating or die coating).

### EXAMPLES

The following describes the invention based on examples, but the invention is not limited to these illustrative examples.

### Example 1

### (Synthesis of Illustration Compound (1))

The illustration compound (1) was synthesized in accordance with the following scheme.

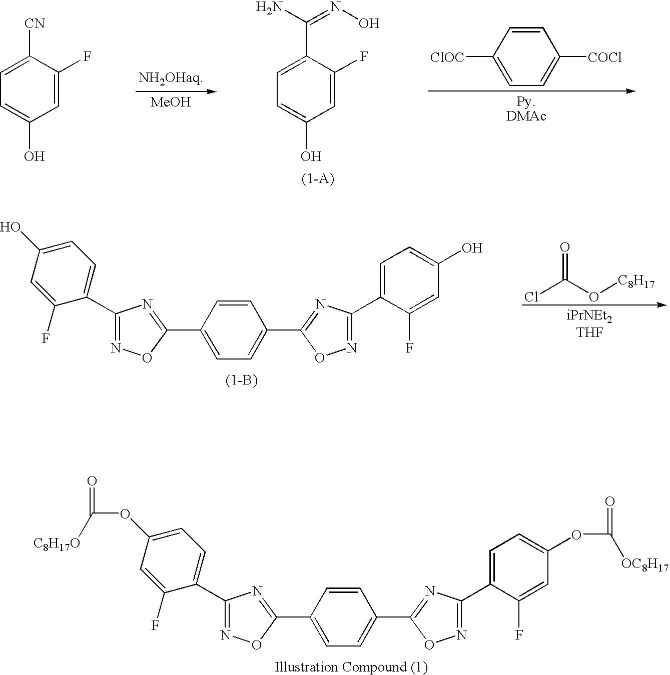

At room temperature, 139 g (2.1 mol) of 50% hydroxylamine aqueous solution was added dropwise to a methanol solution of 144.0 g (1.05 mol) of 2-fluoro-4-cyanophenol. Temperature of the reaction system was gradually increased and stirred under reflux for 3 hours. After the reaction, this was ice-cooled, and cool water was added to the reaction system to effect precipitation of crystals. The thus obtained crystals were collected by filtration and dried to obtain 139.5 g of (1-A) (yield 82%).

A 8.1 ml (0.1 mol) portion of pyridine was added at room temperature to 300 ml of N,N-dimethylacetamide containing 17 g (0.1 mol) of (1-A), and 6.7 g (33 mmol) of phthaloyl dichloride was added thereto in portions. After the addition, this was stirred at room temperature for 30 minutes and then the reaction temperature was increased to 100° C. This was stirred as such for 3 hours and then spontaneously cooled. Since crystals were precipitated when methanol was added thereto, they were collected by filtration. By drying the thus collected crystals, 36 g (yield 83%) of (1-B) was obtained.

At room temperature, 8.7 ml (50 mmol) of N,N-diisopropylethylamine was added to 300 ml of N,N-dimethylacetamide solution of 8.7 g (20 mmol) of (1-B), and 9.6 g (50 mmol) of octyl chloroformate was added dropwise thereto. A 14 g portion of potassium carbonate was added thereto and stirred as such at room temperature for 3 hours. After adding tetrahydrofuran, separation by filtration was carried out, and methanol was added to the resulting filtrate to effect precipitation of crystals. The crystals were collected by filtration and dried, dispersed in methanol, heated and then filtered to obtain 13.4 g of the illustration compound (1) (yield 90%).

Phase transition temperature of the thus obtained illustration compound (1) was measured by DSC measurement and texture observation under a polarization microscope, thereby obtaining the following results.

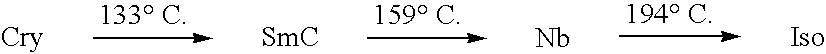

### Example 2

### Synthesis of Illustration Compound (2)

The illustration compound (2) was synthesized in accordance with the following scheme.

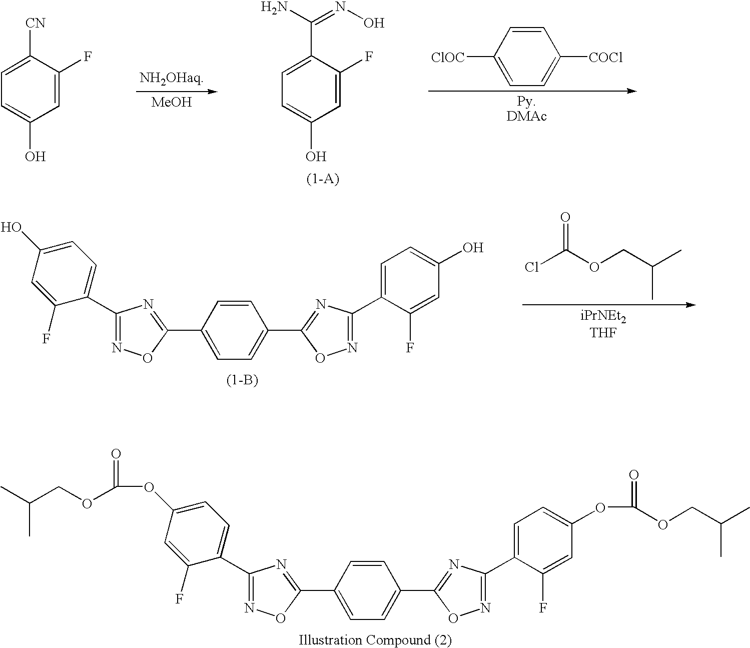

The illustration compound (2) was obtained by the same procedure, except that octyl chloroformate of Example 1 was changed to 2-methylpropyl chloroformate.

Phase transition temperature of the thus obtained illustration compound (2) was measured by DSC measurement and texture observation under a polarization microscope, thereby obtaining the following results.

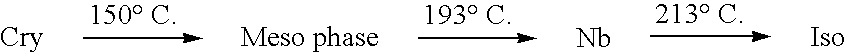

### Example 3

### Synthesis of Illustration Compound (3)

The illustration compound (3) was synthesized in accordance with the following scheme.

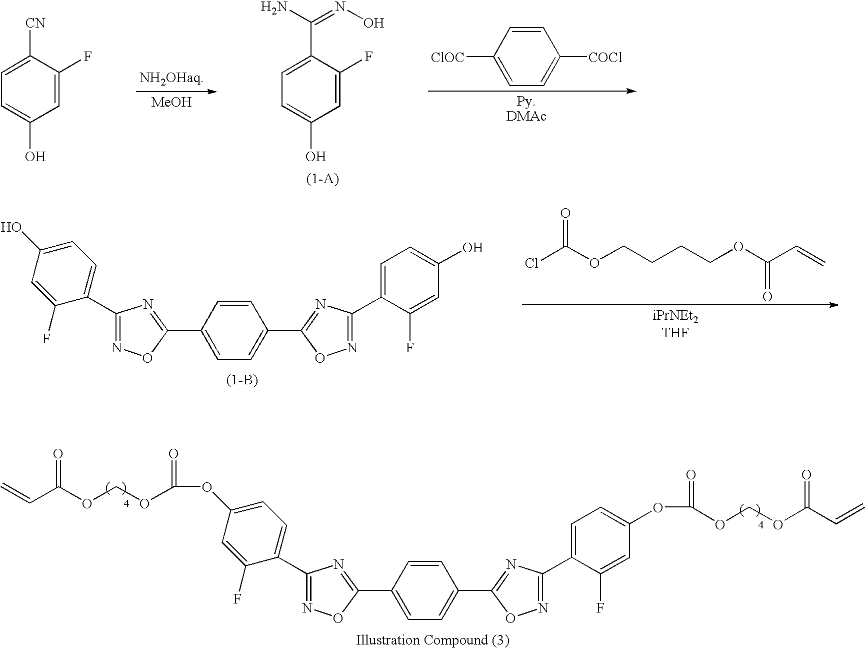

The illustration compound (3) was obtained by the same procedure, except that octyl chloroformate of Example 1 was changed to 4-acryloylbutyl chloroformate.

Phase transition temperature of the thus obtained illustration compound (3) was measured by DSC measurement and texture observation under a polarization microscope, thereby obtaining the following results.

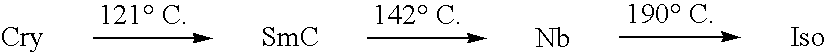

### Example 4

### Synthesis of Illustration Compound (44)

The illustration compound (44) was synthesized in accordance with the following scheme.

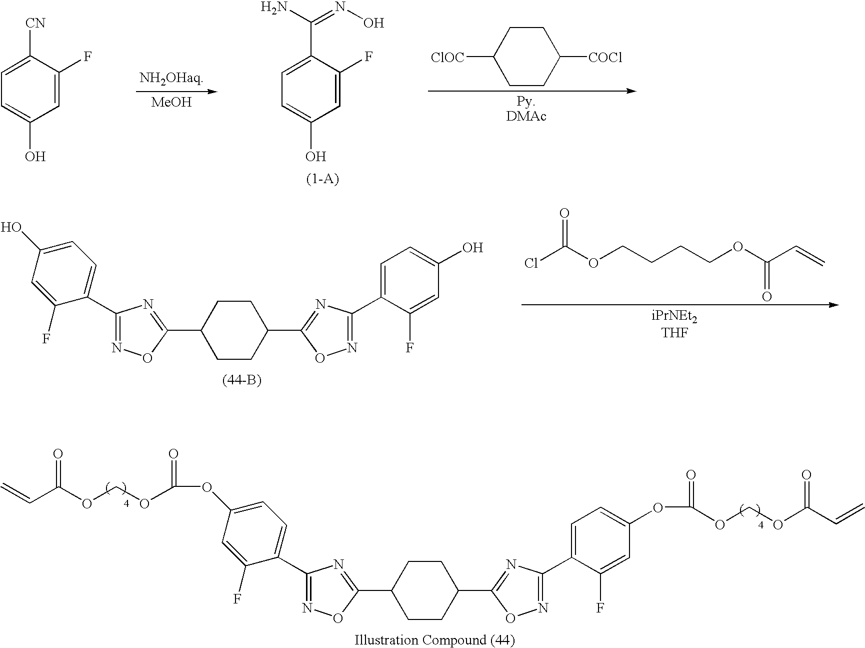

The illustration compound (44) was obtained by the same procedure, except that phthaloyl dichloride of Example 3 was changed to trans-cyclohexyldicarboxylic acid dichloride.

Phase transition temperature of the thus obtained illustration compound (44) was measured by DSC measurement and texture observation under a polarization microscope. At the time of temperature rising, this was transferred from a crystalline phase (Cry) to a nematic phase (N) at around 85° C. and transferred to an isotropic phase (Iso) at around 90° C. At the time of temperature falling, this was transferred to a nematic phase at around 89° C., but phase transfer was not observed thereafter until room temperature.

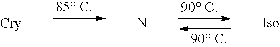

### Example 5

### Verification of Biaxial Property of Illustration Compound (1) 1

The illustration compound (1) was injected into a horizontal orientation cell of 5 μm in cell gap (mfd. by EHC; KSRP-05/A107 MINSS (ZZ)) at 230° C. and allowed to effect homeotropic orientation at 180° C. When measurement of angle-dependency of retardation was carried out under this condition to calculate (nx−nz)/(nx−ny), it was 2.0. In the same manner, it was 2.5 at 170° C. and 3.0 at 165° C. That is, the illustration compound (1) satisfies the relational expression (1).

### Example 6

### Verification of Biaxial Property of Illustration Compound (1) 2

When conoscopic figure of the compound in the cell prepared in the above was observed at 180° C., it showed biaxial property, and it was confirmed that the compound satisfies the relational expression (1). This was the same at 170° C. and 165° C., too.

### Example 7

### Preparation of Retardation Plate

### (Formation of Orientation) Film

A 5% by mass solution was prepared by dissolving a modified polyvinyl alcohol described below and glutaraldehyde (5% by mass of the modified polyvinyl alcohol) in a methanol/water mixed solvent (volume ratio=20/80).

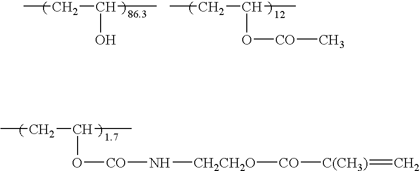

This solution was coated on a glass base and dried with a hot air of 100° C. for 120 seconds, and then an orientation film was formed by carrying out a rubbing treatment. Film thickness of the thus obtained orientation film was 0.5 μm.

### (Formation of Optical Anisotropy Layer)

An optical anisotropy layer application liquid having the following composition was coated on the rubbed orientation film prepared in the above, using a spin coater.

| (Optical anisotropy layer application liquid) | | |
|---|---|---|
| The aforementioned illustration compound (3) | 100.0 | parts by mass |
| The following air interface orientation controlling | 0.2 | part by mass |
| agent V-(1) | | |
| Irgacure 907 (mfd. by Nagase) | 3.3 | parts by mass |
| Chloroform | 700 | parts by mass |
| The following fluoro aliphatic group-containing | 0.5 | parts by mass |
| copolymer (F-1) | | |

Air interface orientation controlling agent V-(1)

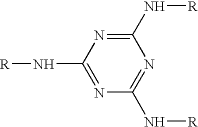

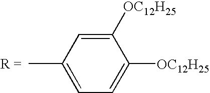

Fluoro aliphatic group-containing copolymer (F-1)

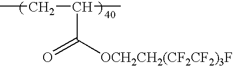

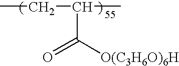

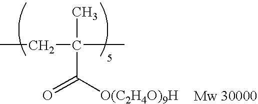

The aforementioned glass base coated with an optical anisotropy layer was put into a constant temperature oven of 130° C. to effect homeotropic orientation of the liquid crystal. Thereafter, orientation condition of the optical anisotropy layer was fixed by irradiating 600 mJ ultraviolet rays. This was spontaneously cooled to room temperature to prepare a retardation plate. Thickness of the optical anisotropy layer was 1.0 μm. The lagging axis was parallel to the rubbing direction.

When measurement of angle-dependency of retardation of the thus obtained retardation plate was carried out, the nx direction was parallel with the glass base face, and the nz direction was parallel against the glass base face. Also, when the (nx−nz)/(nx−ny) was calculated, it was 4.0.

In Example 7, a retardation plate having an optical anisotropy layer in which the refractive index is controlled at a desired value can be provided in the same manner, by replacing the aforementioned illustration compound (3) containing in the aforementioned optical anisotropy layer application liquid optionally with a desired liquid crystal compound of the invention represented by the formulae (1) to (3).

As is evident from Examples 1 to 7, the liquid crystal composition of the invention expresses a biaxial nematic liquid crystal phase. In addition, a retardation plate having an optical anisotropy layer in which the refractive index is controlled at a desired value can be provided using the liquid crystal composition.

It will be apparent to those skilled in the art that various modifications and variations can be made to the described embodiments of the invention without departing from the spirit or scope of the invention. Thus, it is intended that the invention cover all modifications and variations of this invention consistent with the scope of the appended claims and their equivalents.

The present application claims foreign priority based on Japanese Patent Application Nos. JP2005-207143 and JP2005-327001, filed Jul. 15 and Nov. 11 of 2005, respectively, the contents of which are incorporated herein by reference.

1. A liquid crystal compound comprising a region in which an aromatic heterocyclic ring and an aromatic hydrocarbon ring linked together through a single bond, the liquid crystal compound expressing a nematic liquid crystal phase satisfying relational expression (1):
1.1≦(nx−nz)/(nx−ny)≦20
wherein nx, ny and nz represent refractive indexes in three intersecting directions of the nematic liquid crystal phase, nx is the largest refractive index, and nz is the smallest refractive index.

2. The liquid crystal compound according to claim 1, wherein the aromatic heterocyclic ring comprises at least one selected from the group consisting of 1,2,4-oxidiazole ring, 1,3,4-oxadiazole ring, 1,2,4-thiodiazole ring and 1,3,4-thiodiazole ring.

3. The liquid crystal compound according to claim 1, wherein the aromatic heterocyclic ring is 1,2,4-oxadiazole ring.

4. The liquid crystal compound according to claim 1, wherein the liquid crystal compound is a compound represented by one of formulae (1) and (2): 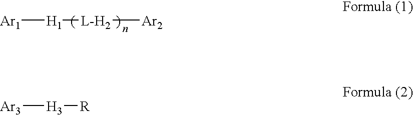
wherein Ar₁ and Ar₂ each independently represent a substituted or unsubstituted phenyl group, biphenyl group or naphthyl group; H₁ and H₂ each represent 1,2,4-oxadiazole ring; L represents a divalent connecting group, and n is an integer of 0 or 1, and
Ar₃ represents a substituted or unsubstituted phenyl group, biphenyl group or naphthyl group; H₃ represents 1,2,4-oxadiazole ring; and R represents a substituted or unsubstituted alkyl group having from 1 to 20 carbon atoms.

5. A liquid crystal compound expressing a nematic liquid crystal phase, which is represented by formula (3): 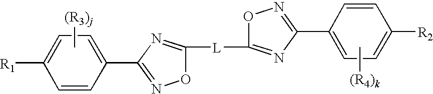
wherein L represents cyclohexyl or methaphenilene; R₁ to R₄ each independently represent a substituent group; and j and k each independently represent an integer of 0 to 4.

6. A liquid crystal composition comprising a liquid crystal compound according to claim 1.

7. A thin film formed from a liquid crystal composition according to claim 6.

8. A liquid crystal display comprising a liquid crystal compound according to claim 1.
